(19) United States
(12) Patent Application Publication
(10) Pub. No.: US 2008/0109033 A1
(43) Pub. Date: May 8, 2008

(54) METHOD AND DEVICE FOR PREVENTION OF PNEUMOTHORAX DURING VASCULAR ACCESS

(75) Inventor: Mehdi Razavi, Houston, TX (US)

Correspondence Address: CONLEY ROSE, P.C.;David A. Rose, P. O. BOX 3267, HOUSTON, TX, 77253-3267, US

(73) Assignee: TEXAS HEART INSTITUTE, Houston, TX (US)

(21) Appl. No.: 11/929,244

(22) Filed: Oct. 30, 2007

Related U.S. Application Data
(60) Provisional application No. 60/863,687, filed on Oct. 31, 2006.
(60) Provisional application No. 60/941,529, filed on Jun. 1, 2007.
(60) Provisional application No. 60/969,370, filed on Aug. 31, 2007.

(51) Int. Cl. A61B 17/03

(52) U.S. Cl. 606/213

(57) ABSTRACT

Methods and devices for prevention of a pneumothorax during vascular access are disclosed herein. The methods utilize a device having a detachable distal portion which serves to block the passageway created after a vascular access procedure. The device may include retention means to secure the device in the passageway. In addition, the methods may make use of tissue adhesives or glues to further ensure sealing of the passage. Furthermore, the disclosed methods and devices may be used to block perforations of blood vessels created during vascular access.

### CROSS-REFERENCE TO RELATED APPLICATIONS

The present application claims the benefit of U.S. Provisional Application Ser. No. 60/863,687, filed Oct. 31, 2006, U.S. Provisional Application Ser. No. 60/941,529, filed Jun. 1, 2007, and U.S. Provisional Application Ser. No. 60/969,370, filed Aug. 31, 2007, all of which are herein incorporated by reference in their entireties for all purposes.

### BACKGROUND

1. Field

This invention relates generally to the field of medical devices. More specifically, the invention relates to an apparatus and a method of preventing pneumothorax during vascular access.

2. Background

Vascular access via use of percutaneous or direct cannulation techniques of the axillary, cephalic, or subclavian venous or arterial systems is subject to multiple complications including the formation of pneumothorax. The lungs are located inside the chest cavity, which is a hollow space. Air is drawn into the lungs by the diaphragm (a powerful abdominal muscle). When the diaphragm contracts, a negative pressure relative to the outside atmosphere is created in the chest cavity, which in turn causes air to flow into the lungs. The pleural cavity is the region between the chest wall and the lungs. If air enters the pleural cavity, either from the outside (open pneumothorax) or from the lung (closed pneumothorax), the lung collapses and it becomes mechanically impossible for the injured person to breathe, even with an open airway. If a piece of tissue forms a one-way valve that allows air to enter the pleural cavity from the lung but not to escape, overpressure can build up with every breath; this is known as tension pneumothorax. It may lead to severe shortness of breath as well as circulatory collapse, both life-threatening conditions. Pneumothorax complications may be caused by an instrument which is used to cannulate the vasculature (most frequently this tool is a hollow needle of variable caliber) inadvertently penetrating the pleural space, thus allowing air to escape into the space. The onset of such a pneumothorax complication due to attempted vascular access is often (but not always) heralded by the return of air (from the lungs) into the syringe connected to the access instrument (or other apparatus connected to the access instrument).

Although the mere entrance into the pleura of the vascular access device does not cause the pneumothorax per se, the subsequent withdrawal of the device which has penetrated the pleural space leads to an open communication passage between the pleural cavity and outside air (or air from the lungs). Frequently, the elasticity of the tissues surrounding this open communication passage are such that the passage does not immediately close on its own, and the passage remains open for minutes, hours or even days. The presence of such an open communication passage allows air from the lungs or outside the patient to be introduced into the pleural space. This process of allowing air introduction is facilitated by the small, normal negative intrapleural pressure. The subsequent accumulation of air causes a pneumothorax (literally air in the thorax), a condition that is clinically undesirable since it can compromise lung function. As the size of the passage, the duration it remains open and the relative pressure difference all vary from case to case, some pneumothoraces/pneumothorax warrant only careful clinical observation to insure that that they do not progress and that they resolve on their own.

Other pneumothoraces/pneumothorax may represent a serious and life threatening complication requiring immediate intervention. In such cases, treatment frequently necessitates the use of a chest tube which provides constant suction for removal of intrapleural air, restoration of normal intrapleural pressure, and the restoration of more complete lung volume and function. Clinically, chest tubes typically are quite uncomfortable for the patient and their use in what would be otherwise uncomplicated clinical procedures almost invariably leads to extra days of hospitalization and numerous significant extra costs to the ultimate payers of the medical expenses. Introduction of chest tubes also is reported to increase the risk of other complications; including infection, bleeding, and/or blood clots. Overall, the incidence of death from iatrogenic pneumothorax approaches 20 percent. Avoidance of pneumothoraces is thus highly desirable for multiple clinical and for economic reasons.

Consequently, there is a need for improved methods and devices for preventing pnuemothorax during vascular access.

### BRIEF SUMMARY

Methods and devices for prevention of a pneumothorax during vascular access are disclosed herein. The methods utilize a device having a detachable distal portion which serves to block the passageway created after a vascular access procedure. The device may include retention means to secure the device in the passageway. In addition, the methods may make use of tissue adhesives or glues to further ensure sealing of the passage. Furthermore, the disclosed methods and devices may be used to block or fill perforations or holes in blood vessels created during vascular access. Other aspects and advantages of the disclosed devices and methods will be described in more detail below.

In an embodiment, a method of preventing pneumothorax in a patient comprises inserting a device having a outer hollow member and an inner cannula into a lung of the patient to create access to the lung. The outer hollow member has an open distal end and the inner cannula comprises a proximal portion and a detachable distal portion. The method further comprises extending the detachable portion of the inner cannula past the open distal end of the outer hollow member. In addition, the method comprises detaching the detachable distal portion from the proximal portion to prevent pneumothorax in the patient.

In an embodiment, a device for the prevention of pneumothorax comprises an inner cannula having a distal portion and a proximal portion. The cannula has a means for detaching the distal portion from the proximal portion. The distal portion comprises a plurality of openings from injecting a tissue adhesive. In addition, the device comprises an outer hollow member having an open distal end. The inner cannula is slidably disposed coaxially within the outer hollow member.

In another embodiment, a device for the prevention of pneumothorax comprises a hollow inner cannula having a distal portion and a proximal portion. The cannula has a means for detaching distal portion from the proximal portion and the distal portion comprises a plurality of openings for injecting a tissue adhesive. The device also comprises one or more retainment members disposed on the distal portion. The one or more retainment members have an expanded position and a contracted position. Additionally, the device comprises an outer hollow member having an open distal end. The inner cannula is slidably disposed coaxially within the outer hollow member

In another embodiment, a method of filling a perforation in a blood vessel comprises inserting a device having an outer hollow member and an inner cannula into the perforation. The outer hollow member has an open distal end and the inner cannula comprises a proximal portion and a detachable distal portion. The method also comprises extending the detachable portion of the inner cannula past the open distal end of the outer hollow member. The method additionally comprises detaching the detachable distal portion from the proximal portion to fill the perforation in the blood vessel.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIGS. 1A-G illustrate embodiments of a device for preventing pneumothorax;

FIGS. 2A-B illustrate different embodiments of a device for preventing pneumothorax;

FIGS. 3A-F illustrates an embodiment of a method for preventing pneumothorax; and

FIG. 4 illustrates another embodiment of a method for preventing pneumothorax.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”.

### DETAILED DESCRIPTION OF PREFERRED EMBODIMENTS

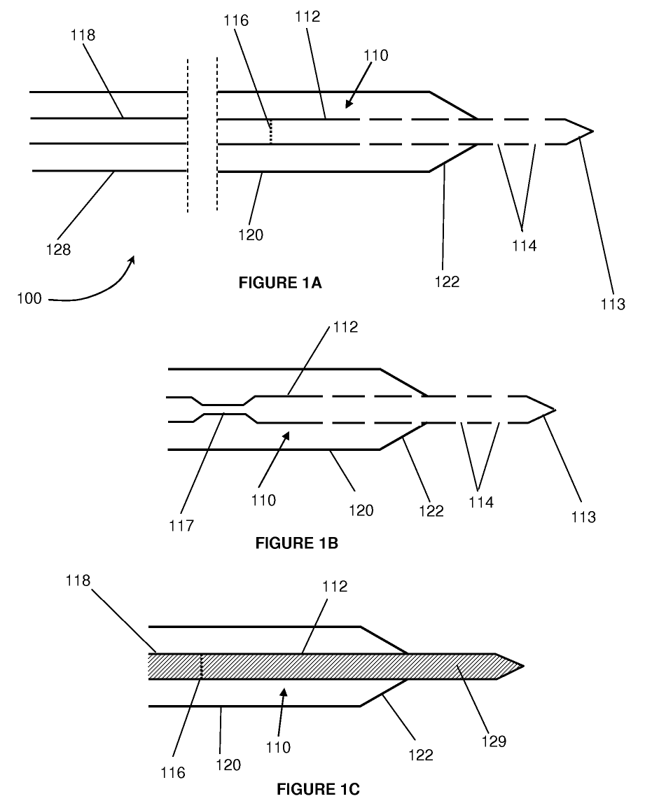

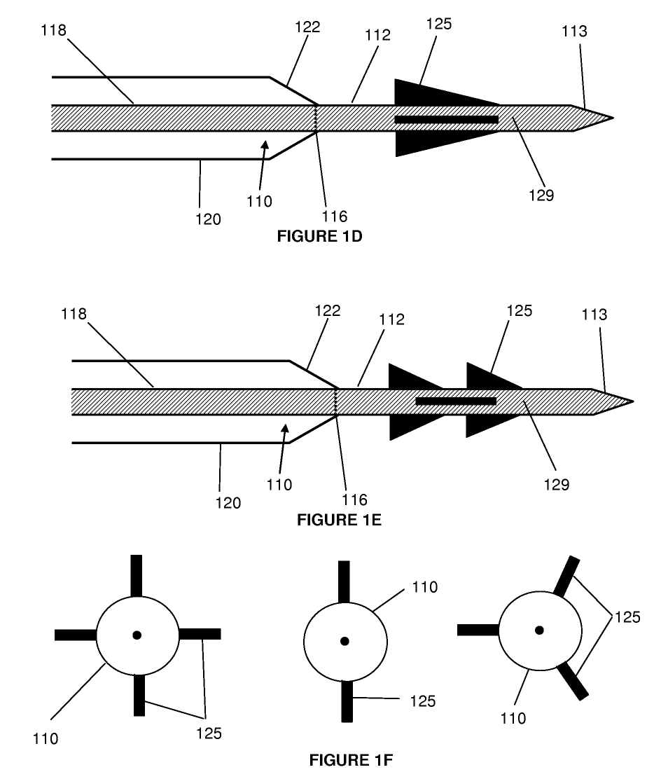

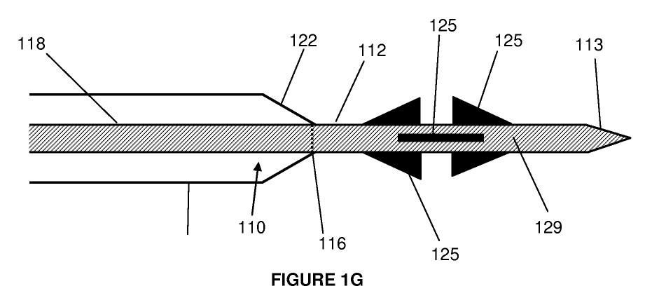

FIGS. 1A-G illustrate various embodiments of a device 100 for preventing a pneumothorax during vascular access. Generally, embodiments of the device 100 comprise an outer hollow member 120 and an inner cannula 110. Outer hollow member 120 comprises a sharpened distal end 122 which is preferably open. Inner cannula 110 is movably disposed coaxially within outer hollow member 120. In addition, inner cannula 110 comprises a detachable distal portion 112. Detachable distal portion 112 may be detached in a variety of ways which will be described in more detail below. Accordingly, inner cannula 110 may comprise a means 116 for detaching distal portion 112 from proximal portion 118. The detachable distal portion 112 serves to block the passage created by a vascular access device and prevent air from entering the passage so as to prevent pneumothorax. The detachable distal portion 112 preferably comprises one or more ports or openings 114 for injecting an adhesive or a sealant into the passage created by the vascular access device. Furthermore, distal portion 112 may comprise any number of openings 114 with a variety of patterns. Openings 114 may be circular, oval, slits, or combinations thereof.

Outer hollow member 120 typically comprises a tubular or needle-like configuration. Generally, distal end 122 of outer hollow member 120 is open and may comprise a sharpened tip to penetrate tissue. In some embodiments, distal portion 122 of outer hollow member 120 may comprise a plurality of openings for the ejection of a tissue adhesive. The openings in the outer hollow member 120 may be aligned with openings 114 in the distal portion 112 of inner cannula 110. It is also envisioned that outer hollow member 120 may be an existing device used for vascular access such as catheters, trocars, biopsy needles, drains, and the like.

Outer hollow member 120 may comprise any suitable biocompatible material such as metals, polymers, or combinations thereof. Examples of suitable metals include without limitation, surgical steel. Examples of suitable polymers include without limitation, polycarbonate, polyethylene, polypropylene, or combinations thereof. In an embodiment, outer hollow member 120 comprises a metal syringe needle. In addition, outer hollow member 120 may comprise any suitable outer diameter. More particularly, outer hollow member 120 may have an outer diameter ranging from about 0.1 cm to about 1.5 cm, alternatively from about 0.5 cm to about 1 cm.

The cannula 110 may comprise a distal segment or portion 112. Preferably, the inner cannula 110 is hollow. In addition, the tip 113 of distal portion may be sharpened to facilitate penetration of the device into tissue. Distal tip 113 may optionally have an opening for injection of drugs or sealant into vascular tissue. In the embodiment depicted in FIG. 1C, inner cannula 110 comprises a solid-bore filling 129. In other words, in such an embodiment, cannula 110 is not hollow, but is solid throughout its length.

In an embodiment, the cannula 110 may comprise a means of detachment 116 between distal portion 112 and proximal portion 118 of cannula 110 as shown in FIGS. 1A-B. The means of detachment 116 may comprise a mechanical means such as a groove, a narrowed portion (e.g., a waist), a threaded connection, snap-fit connection or other such means which enables simple detachment, disconnection or amputation of the distal segment with subsequent retention of the detached distal portion 112 in the body. Accordingly, the detachable distal segment 112 of the cannula 110 may serve as a means of blocking or sealing an undesirable air passageway created by the device 100.

The detachment may be facilitated a weakened section of cannula 110 between the distal 112 and proximal 118 segments. Furthermore, the detachment may be assisted by means of retainment that serve to anchor the distal segment such that it resists being retracted from the body tissue, and/or by features which assist in the transfer and concentration of axial and/or torsional forces to a localized region of the cannula between the distal and proximal segments. This detachment may be facilitated by cutting the retained distal portion 112 of the cannula 110 from the proximal portion 118, which would generally be performed so that the retained distal portion 112 is solely in the tissue and no portion of the cannula 110 remains protruding from the tissue. Cutting the distal portion 112 may be accomplished by using mechanical devices such as scissors, knives, clamps, etc. Alternatively, distal portion 112 may be removed using application of electricity or heat to remove detach distal portion 112.

Referring to FIG. 1B, the inner cannula 110 may comprise a narrowed portion 117 when compared to the other portions of the cannula 110 (e.g., a waist portion) as a means of detaching distal portion 112 from proximal portion 118. The waist 117 may be disposed at any length of the cannula 110. More specifically, the waist 117 may be located at a distance ranging from about 0.1 cm to about 3 centimeters proximal to the distal tip, alternatively from about 0.25 cm to about 2 cm, alternatively from about 0.5 cm to about 1 cm. Waist or narrowed portion 117 may undergoing steps during manufacturing which reduce its tensile and/or torsional strength (as compared to the remainder of the cannula), such as perforations or scoring.

Referring now to FIGS. 1D-F, in a further embodiment, distal portion 112 cannula 110 may be configured with a retainment means such as one or more retainment members 125 as shown in FIGS. 1D-F. The one or more retainment members 125 preferably comprise a contracted position and an expanded position. In the contracted position, retainment members 125 are either folded flush with the outer surface of cannula 110 or may retract into the interior or lumen of cannula 110. When distal portion 112 is extended beyond distal end 122 of outer hollow member 120, retainment members 125 may expand into an expanded position as shown in FIG. 1D. Accordingly, retainment members 125 may be spring loaded in their contracted position such that once distal portion 112 is extended beyond the distal end 122, they automatically expand into their expanded position. In the expanded position, retainment members 125 may either prevent movement of distal portion 112 in a proximal direction or retainment members 125 may impinge on the inner surfaces of the passageway. Retainment members 125 create obstruction and resistance points, so that the distal portion 112 cannot be easily retracted back into the outer hollow member 120. Thus, the expanded position of retainment members 125 results in retainment of the distal portion 112 of the cannula 110 in the body.

Retainment members 125 may have a variety of different configurations. FIG. 1E illustrates retainment members 125 in a triangular configuration. In other embodiments, retainment members 125 may comprises barbs, protrusions, spikes, tines, or combinations thereof. Retainment members 125 may be arranged in any suitable configuration around distal portion 112 of cannula 110. FIG. 1F illustrates axial views of various configurations of retainment members 125. Thus, retainment members 125 may be disposed in an aligned parallel configuration or an offset configuration. Optionally, additional more proximal sets of retainment members 125 may be disposed on the distal portion 112. These addition retainment members 125 may be aligned similarly to the first set of retainment members 125, or in the reverse direction as shown in FIG. 1G. These additional retainment members may provide additional stabilization to distal portion 112 in the passageway.

In alternative embodiments, retainment member 125 may comprise a coating of adhesive or glue on the outer surface of distal portion 112 in addition to adhesive or glue that is ejected from openings 114. Furthermore, surface of distal portion 112 may be texturized to provide friction and thus, further retainment of distal portion 112 in passageway. For example, outer surface of distal portion 112 may comprise a diamond pattern knurling for improved retainment of distal portion 112 in tissue.

Preferably, the cannula 110 performs its designed functions in combination with an adhesive 161 or sealant or other glue-like substance which is caused to be transferred through the hollow cannula and is thereby present in the passage in the peri-pleural tissue created by the device 100.

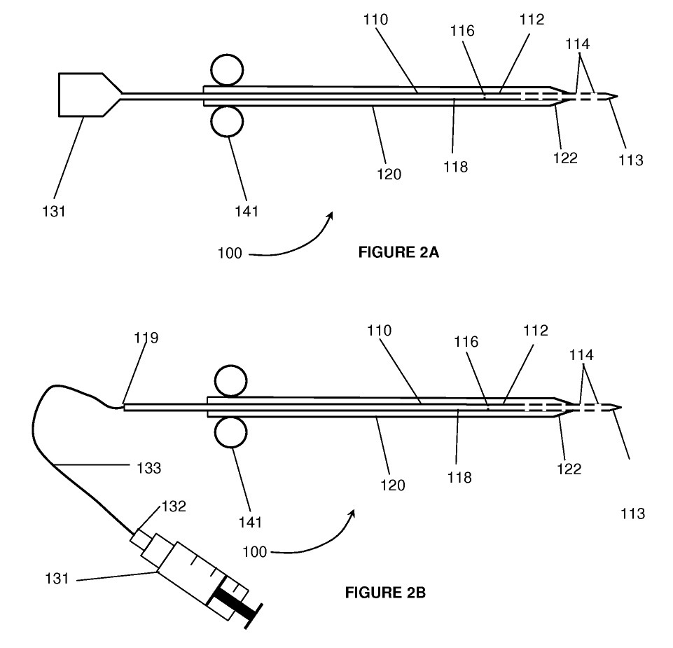

Referring now to FIGS. 2A-B, a reservoir 131 containing an adhesive or sealant may be coupled to the proximal end of inner cannula. The cannula 110, on its proximal end and in the case of the cannula 110 being hollow, is in fluid connection to the reservoir or applicator-container 131 (such as a syringe) containing a sealant or glue. The reservoir 131 may be integral to inner cannula 110 or it may be detachably coupled to the proximal end of cannula 110 such as using a threaded connection. According to one embodiment, inner cannula 110 may be in fluid connection with a syringe as shown in FIG. 2B. That is, a flexible conduit 133 may be connected to the proximal end 113 of inner cannula 110 and then connected to the outlet 132 of a syringe. As shown in FIGS. 2A-B, the proximal portion 128 of outer hollow member 120 may also comprise a handle 141 for removing the outer hollow member 120 from inner cannula 110. Alternatively, proximal end of outer hollow member 120 may be coupled to a syringe for the injection of drugs or other substances into the patient.

In embodiment, cannula 110 is made of a biocompatible material. Examples of suitable materials include without limitation, polyurethane, polysilicone, rubber, polyethylene, polypropylene, polycarbonate, polytetrafluoroethylene, polyethylene glycol, or combinations thereof. In addition, the cannula 110 may be made of a biodegradable and/or bioresorbable material which allows retention of the distal portion 112 in the body indefinitely or the time necessary for the degradation and/or absorption by the body (in the case of biodegradable or bioabsorbable). Examples of biodegradable materials include without limitation, polyglycolic acid, polylactic acid, polycaprolactone, polypropylene fumarate, polyanhydrides, polyphosphazenes, or combinations thereof.

Inner cannula 110 may be manufactured with any suitable length and diameter. In particular, inner cannula my have a diameter ranging from about 0.1 mm to about 50 mm, alternatively from about 0.2 mm to about 25, alternatively from about 0.1 mm to about 20 mm. As such, the disclosed devices may be matched to specific patient's needs and/or to different devices used initially for attempted vascular access. The outer diameter of the inner cannula 110 is preferably less than the inner diameter of the outer hollow member 120 which is used to obtain vascular access.

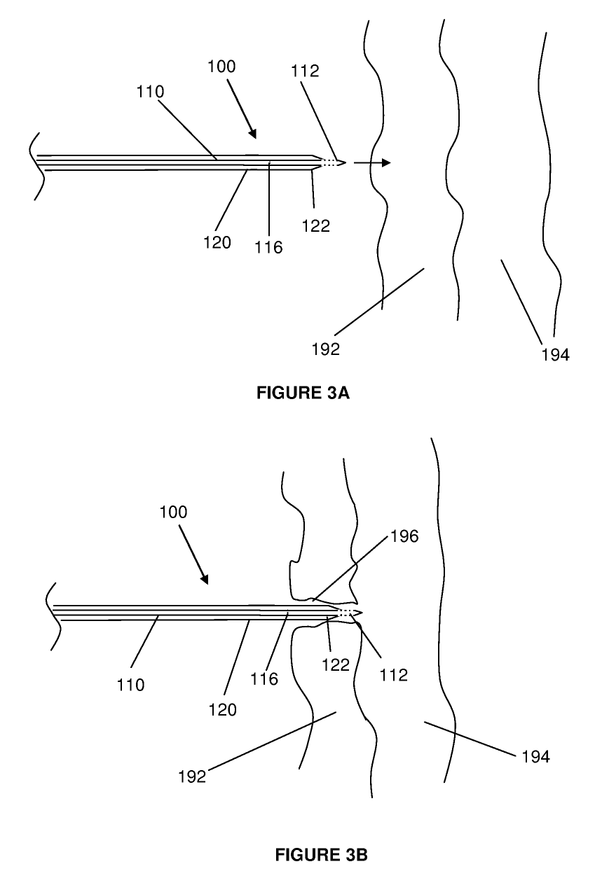

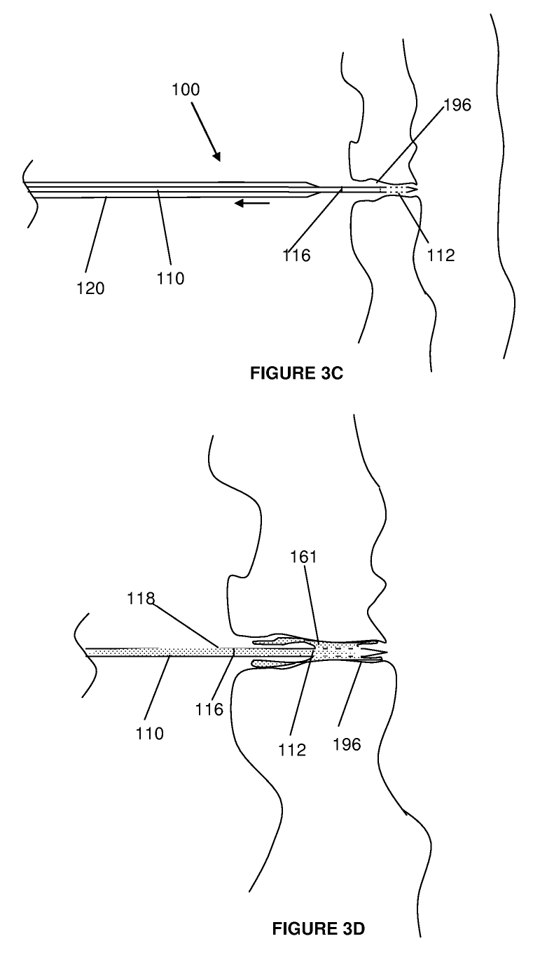

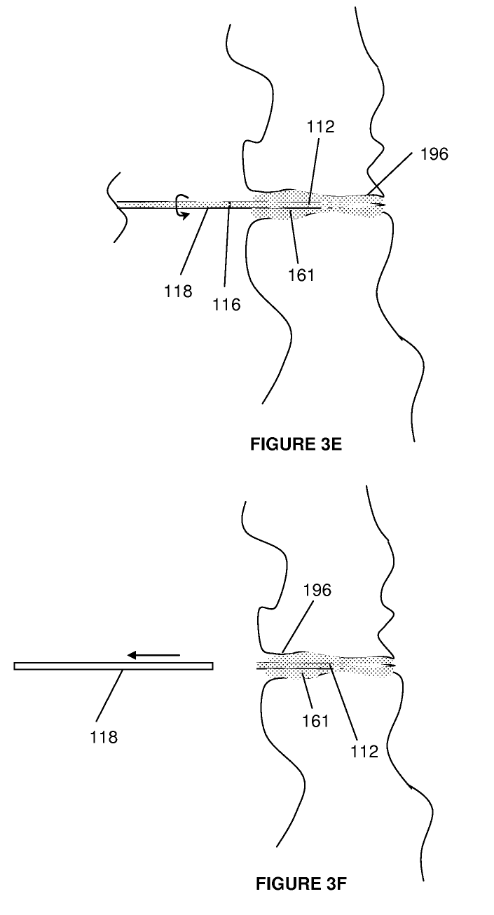

Embodiments of a method of preventing pneumothorax utilize the devices described in detail above. Referring to FIGS. 3A-D, outer hollow member 120 may be inserted into a patient to access tissue such as lung tissue 192. The device 100 may penetrate through tissue 192 into pleural space 194. The inner cannula 110 may already be disposed within outer hollow member 120 or alternatively, inner cannula 110 may be inserted into outer hollow member after insertion of outer hollow member 120 into the lung tissue. Inner cannula 110 may be advanced such that its distal tip 113 extends beyond the distal end 122 of outer hollow member 120 as shown in FIG. 3C. At this point, a tissue adhesive 161 or glue may be injected through inner cannula 110 and out the openings to seal the passageway 196 as shown in FIG. 3D. The container or reservoir 131 may be compressed in volume allowing the tissue adhesive it contains to travel to the distal portion 112 of the cannula 110, and eventually for the tissue adhesive to extrude into passageway 196 via the openings 114. Time is allowed to pass to allow the tissue adhesive to seal and/or to coagulate similar to a glue. The cannula 110 acts as a conduit to distribute the glue or liquid formation to the internal site of the undesirable air passage that could cause the pneumothorax. Outer hollow member 120 may then be retracted leaving distal portion 112 of inner cannula 110 positioned within the passage created by the outer hollow member 120.

Generally, the tissue adhesive 161 is a biocompatible substance which will not invoke an immune reaction in the body. Examples of suitable substances include without limitation, acrylic glue, polymethyl methacrylate, fibrin glue, hydrogels, cyanoacrylate glue, proteins, polysaccharides, gelatin, or combinations thereof. However, any suitable sealant or glue known to those of skill in the art may be used. Preferably, the sealant or glue is a liquid of sufficiently high viscosity that it may be transferred through the hollow cannula without undue pressure or force. Furthermore, adhesive 161 may comprise one or more drugs which may provide therapy to the accessed tissue regions. Examples of suitable drugs include without limitation, antibiotics, anti-clotting drugs, heparin, anti-inflammatory drugs, steroids, talc, clotting drugs, or combinations thereof.

Referring now to FIGS. 3E-F, once the outer hollow member 120 is outside of the relevant tissue (e.g. pleura), the distal portion 112 of the cannula 110 may be detached from the proximal portion 118, leaving distal portion 112 retained in the passageway 196 to prevent air from entering and thus, preventing pneumothorax. Distal portion 112 may be detached using a number of techniques, depending on the means of detachment incorporated into the device. For example, if the means of detachment comprises scoring of the inner cannula 110, an external cutting device may be used to sever the distal portion 112 from the proximal portion of inner cannula. In other embodiments, motion of the proximal portion 118 may transmit either axial or torsional forces (or both types of forces simultaneously) to inner cannula 110 as shown in FIG. 3E. Proximal portion 118 of cannula 110 may be pulled in the proximal direction, and thus snapping the proximal portion 118 from the distal portion 112 of the cannula 110. As such, proximal portion may be removed from distal portion through a variety of motions such as pulling or twisting of the proximal portion.

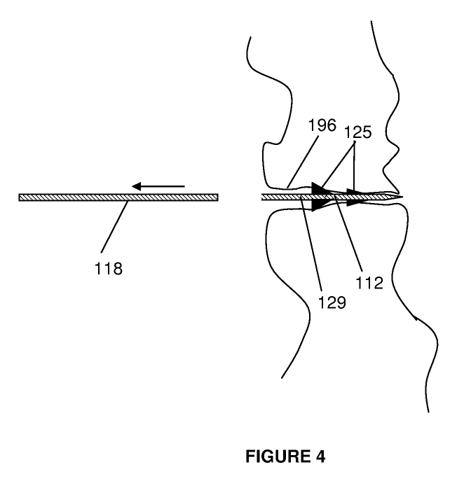

In some cases, the method does not require the use of an adhesive or glue as shown in FIG. 4. In other words, the inner cannula 110 may be first be inserted through the outer hollow member 120 into the target tissue site and then the surgeon or user may simply detach the distal portion 112 of cannula 110 without dispensing or injecting a tissue adhesive 161 or glue into the passageway, leaving the distal portion 112 as the exclusive means to seal the passage created by the device 100. Preferably, in such embodiments, inner cannula 110 comprises a solid bore 129 as shown in FIG. 4 to completely block any inlet of air into the passageway 196.

Several different methods may be employed for fixing or retaining the distal segment in the peri-pleural tissue 192. By the operator pulling back or rotating the proximal side of the cannula, this “waist” separates, without retracting the distal segment of the cannula which has been stabilized by the aforementioned tines. As described in detail above, the device 100 may comprise a means of retention such as without limitation, one or more retention members 125. Retention members 125 create obstruction and resistance points so that the distal portion 112 cannot be easily retracted back from tissue 192. Proximal portion 118 may be removed leaving distal portion 112 of the cannula 110 in the body.

In another embodiment, cannula 110 may be placed over a wire which is then introduced through the outer hollow member 120. In this example, wire may act as a guide wire for cannula 110. Outer hollow member 120 may then be withdrawn, leaving the wire and cannula 110 in place. The wire is then withdrawn from the cannula leaving only the cannula in place in the tissue.

Both the cannula 110 and tissue adhesive 161 may be composed of non-inflammatory or bioresorbable or biodegradable material which ultimately will be absorbed and/or dissipated by the patient's body. These may also be made of a wide range of other biologically inert and benign materials. Using the above methods, devices and materials eliminates the (potential) air passage 196 between the pleural space 194 and air outside of the patient that is created as the device 100 is retracted. This, in turn, prevents formation of a pneumothorax. Because the total economic costs associated with the management of pneumothorax/pneumothoraces are so high, this invention can be practiced in all cases with a suspected increased risk of pneumothorax, while still delivering a net cost saving to the system compared to the present systems for managing such complications.

In a further embodiment, it is contemplated that the disclosed device 100 may be used to plug or block perforations or accidental penetration of blood vessels (i.e. veins or arteries). In the course of vascular access, very frequently other vessels in which access is not intended can be traversed, penetrated, punctured, or entered via the access device (usually a needle or similar hollow bore device). Such inadvertent puncture can lead to bleeding, oozing, or other unwanted communications which can additionally have unwanted long-term sequelae. Such sequelae include but are not limited to bleeding, arterio-venous fistula (that is, a communication between arterial and venous vessels), pseudoaneurysms (disruptions in the vessel wall without complete closure of the wall), and dissections (disruption of the vessel wall and creation of a tract within the layers of the vessel wall). Thus, if a surgeon discovers that a blood vessel has been inadvertently punctured, any of the embodiments of the disclosed device 100 may be inserted at the puncture site such that distal portion 112 fills the perforation. Using any of the methods described above, the surgeon may detach proximal portion from distal portion 112, leaving distal portion 112 to create a plug and prevent bleeding from the puncture site or perforation. In addition, tissue adhesive may be dispensed through embodiments of the device 100 having openings 114 to further secure distal portion 112 at the vessel puncture site.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of a reference in the Description of the Related Art is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. A method of preventing pneumothorax in a patient comprising:
a) inserting a device having an outer hollow member and an inner cannula into a lung of the patient to create access to the lung, the outer hollow member having an open distal end, wherein the inner cannula comprises a proximal portion and a detachable distal portion;
b) extending the detachable portion of the inner cannula past the open distal end of the outer hollow member; and
c) detaching the detachable distal portion from the proximal portion to prevent pneumothorax in the patient.

2. The method of claim 1 wherein (a) comprises inserting the outer hollow member into the lung first and then inserting the inner cannula into the lumen of the outer hollow member.

3. The method of claim 1 further comprising retracting the outer hollow member after (b).

4. The method of claim 1 wherein the inner cannula is hollow and comprises a plurality of openings at the detachable distal portion for injecting a tissue adhesive.

5. The method of claim 1 further comprising injecting a tissue adhesive through the plurality of openings and allowing the tissue adhesive to cure after (b) and before (c).

6. The method of claim 5 wherein the tissue adhesive comprises a drug selected from the group consisting of antibiotics, anti-clotting drugs, heparin, anti-inflammatory drugs, steroids, or combinations thereof.

7. The method of claim 1 wherein the inner cannula comprises a solid bore.

8. The method of claim 1 wherein (c) comprises twisting the proximal portion to detach the detachable distal portion.

9. The method of claim 1 wherein (c) comprises cutting the inner cannula to detach the detachable distal portion.

10. The method of claim 1 wherein the distal portion comprises one or more retainment members, wherein each retainment member has an expanded position and a contracted position.

11. The method of claim 10 further comprising expanding the one or more retainment members to secure the distal portion.

12. A device for the prevention of pneumothorax comprising:
an inner cannula having a distal portion and a proximal portion, said cannula having a means for detaching distal portion from said proximal portion, wherein said distal portion comprises a plurality of openings for injecting a tissue adhesive; and
an outer hollow member having an open distal end, wherein said inner cannula is slidably disposed coaxially within said outer hollow member.

13. The device of claim 12 wherein the distal portion comprises a means to retain distal portion in biological tissue.

14. The device of claim 13 wherein said means to retain distal portion in biological tissue comprises one or more retainment members disposed on the outer surface of said distal portion.

15. The device of claim 13 wherein said means to retain distal portion in biological tissue of coated comprises a coating of tissue adhesive around said distal portion.

16. The device of claim 13 wherein said inner cannula comprises a biodegradable material.

17. The device of claim 12 further comprising a reservoir for containing a tissue adhesive in fluid connection with said inner cannula.

18. The device of claim 12 wherein said means for detaching distal portion from said proximal portion comprises a snap-fit connection, a threaded connection, a narrowed waist portion between the proximal portion and the distal portion, or perforations between the distal portion and the proximal portion.

19. A device for the prevention of pneumothorax comprising:
a hollow inner cannula having a distal portion and a proximal portion, said cannula having a means for detaching distal portion from said proximal portion, wherein said distal portion comprises a plurality of openings for injecting a tissue adhesive;
one or more retainment members disposed on said distal portion, wherein said one or more retainment members have an expanded position and a contracted position; and
an outer hollow member having an open distal end, wherein said inner cannula is slidably disposed coaxially within said outer hollow member.

20. The device of claim 19 wherein said means for detaching distal portion from said proximal portion comprises a snap-fit connection, a threaded connection, a narrowed waist portion between the proximal portion and the distal portion, or perforations between the distal portion and the proximal portion.

21. A method of filling a perforation in a blood vessel comprising:
a) inserting a device having an outer hollow member and an inner cannula into the perforation, the outer hollow member having an open distal end, wherein the inner cannula comprises a proximal portion and a detachable distal portion;
b) extending the detachable portion of the inner cannula past the open distal end of the outer hollow member; and
c) detaching the detachable distal portion from the proximal portion to fill the perforation in the blood vessel.

22. The method of claim 21 wherein the inner cannula is hollow and comprises a plurality of openings at the detachable distal portion for injecting a tissue adhesive.

23. The method of claim 21 further comprising injecting a tissue adhesive through the plurality of openings and allowing the tissue adhesive to cure after (b) and before (c).

24. The method of claim 21 wherein the distal portion comprises one or more retainment members, wherein each retainment member has an expanded position and a contracted position.

25. The method of claim 24 further comprising expanding the one or more retainment members to secure the distal portion in the vessel.
